(19) United States
(12) Patent Application Publication
(10) Pub. No.: US 2022/0065852 A1
(43) Pub. Date: Mar. 3, 2022

(54) DEVICES AND METHODS FOR DETECTING VIRAL INFECTION

(71) Applicant: Maxim Biomedical, Inc., Rockville, MD (US)

(72) Inventors: Mei-Jhy Su, North Potomac, MD (US); Juehn-Shin Maa, North Potomac, MD (US); Jonathan Maa, North Potomac, MD (US); Jessica Maa, North Potomac, MD (US); Julia Maa, North Potomac, MD (US)

(21) Appl. No.: 17/461,264

(22) Filed: Aug. 30, 2021

Related U.S. Application Data
(60) Provisional application No. 63/071,653, filed on Aug. 28, 2020.

(51) Int. Cl. G01N 33/543; G01N 33/569

(52) U.S. Cl. CPC G01N 33/54388; G01N 2469/10; G01N 2333/165; G01N 33/56983

(57) ABSTRACT

Methods and devices for the detection of coronavirus infection are disclosed. The methods include detecting the presence of coronavirus nucleocapsid protein comprising the use of antibodies bound to nanoparticles immobilized on a solid support chromatographic immunoassay. The devices and methods include detection of SARS-CoV-2.

### TECHNICAL FIELD

Embodiments of the present disclosure relate generally to methods and devices for detecting viral infection and, in particular, systems and methods comprising detection of coronavirus.

### BACKGROUND OF THE INVENTION

Coronaviruses are a type of virus. There are many different kinds, and some cause disease. A newly identified coronavirus, SARS-CoV-2, has caused a worldwide pandemic of respiratory illness, called COVID-19. COVID-19 is highly infectious: as of August 2020 approximately 25 million global cases were recorded, and almost 850,000 deaths were attributed to the disease; as of August 2021, 215 million global cases were recorded, and over 4.5 million deaths have now been attributed to the disease.

Coronaviruses (CoV) are a family of viruses that cause a variety of illnesses, which vary from a mild cold to severe diseases like Severe Acute Respiratory Syndrome (SARS). This family of viruses are zoonotic (transmitted between animal and human) and several have already been identified in animals but have yet to infect humans. The COVID-19 disease was first identified in Wuhan, China in December 2019.

Coronaviruses are named for the crown-like spikes on their surface. There are four main sub-groupings of coronaviruses, known as alpha, beta, gamma, and delta. Human coronaviruses were first identified in the mid-1960s. The seven coronaviruses that can infect people are: 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS) and SARS-CoV-2 (the novel coronavirus that causes coronavirus disease 2019, or COVID-19).

Most people infected with the COVID-19 virus experience mild to moderate respiratory illness and recover without requiring special treatment. Older people, and those with underlying medical problems like cardiovascular disease, diabetes, chronic respiratory disease, and cancer are more likely to develop serious illness.

Development of strategies for combatting the COVID-19 pandemic are ongoing, and an integral part of our ability to manage the disease is to prevent and slow down transmission. It is readily apparent that detecting infection, tracking spread, and enabling contact tracing are vital approaches for bringing the disease under control.

The COVID-19 virus spreads primarily through droplets of saliva or discharge from the nose when an infected person coughs or sneezes, and accordingly, it is important that individuals practice respiratory etiquette (for example, by coughing into a flexed elbow or by wearing a face mask). Authorities also recommend that individuals protect themselves and others from infection by frequent handwashing, using an alcohol based rub frequently, and not touching one's face.

At this time, there are a few vaccines and treatments for COVID-19. However, there continues to be ongoing research and clinical trials to improve currently available therapeutics. In particular, there is ongoing research to develop vaccines and treatments that may combat emerging variants of SARS-CoV-2.

As the number of cases and deaths continue to rise daily, it is imperative that a multi-pronged approached be employed to manage the COVID-19 crisis. In addition to treating symptoms and developing preventative vaccines, it is crucial that effective diagnostic and detection mechanisms be designed and deployed in order to enable both early detection (therefore treatment at early stages of infection) and also to prevent the spread of the infection.

Accordingly, there is a need for rapid and accurate detection methods and devices for identifying SARS-CoV-2 infection and agents that cause COVID-19.

### SUMMARY OF THE INVENTION

In an embodiment, the present disclosure relates to detection methods and devices for identifying SARS-CoV-2 infection and agents that cause COVID-19.

In an embodiment, the present disclosure relates to detection methods and devices comprising a rapid test enabling the qualitative detection of antigens indicative of SARS-CoV-2 infection and used as an aid for diagnosis of COVID-19.

In other embodiments, the present disclosure provides test kits comprising a dipstick test strip, sample buffer tube, tube stand, sample buffer ampoule, nasopharyngeal or nasal swab, pipette, label sheet and instruction guide.

In certain embodiments, the present disclosure provides methods comprising the use of a nasopharyngeal or nasal swabs to obtain a sample from a subject requiring an assessment concerning coronavirus infection, utilizing the swabbed material in a lateral flow assay and determining the presence of a coronavirus infection by the detection of a visual signal.

In other embodiments, the present disclosure provides uses of a novel lateral flow assay enabling the visual detection of a signal to indicate the presence or absence of a SARS-CoV-2 infection.

### BRIEF DESCRIPTION OF FIGURES

FIG. 1 provides a summary of a nasopharyngeal or nasal swab testing procedure.

FIG. 2 provides a summary of VTM testing procedure.

FIG. 3 provides a graphical depiction of result interpretation.

### DETAILED DESCRIPTION

The following detailed description is exemplary and explanatory and is intended to provide further explanation of the present disclosure described herein. Other advantages, and novel features will be readily apparent to those skilled in the art from the following detailed description of the present disclosure. Texts and references mentioned herein are incorporated in their entirety, including U.S. Patent Application Ser. No. 63/071,653 filed on Aug. 28, 2021.

The term “subject” should be construed to include subjects, for example medical or surgical subjects, such as humans and other animals suffering from viral infection.

Coronavirus disease 2019 (COVID-19) is an infectious disease caused by severe acute respiratory syndrome coronavirus 2 (SARS-CoV-2), and has emerged as one of the most widespread and devastating pandemics in the recorded history of mankind. According to the World Health Organization, globally more than 215 million documented infections and more than 4.5 million deaths have resulted as a consequence of SARS-CoV-2 as of August 2021. The true incidence of the infection however is largely underestimated, since in most countries asymptomatic and paucisymptomatic people are tested only if they come in direct contact with sick patients or belong to at-risk subgroups. There exists a critical need therefore, for accurate and reliable testing methods in order to identify and contain the spread of infection.

Immense efforts have been undertaken by the scientific community to visualize and understand the complex biology driving the pandemic through structure—function studies of different SARS-CoV-2 proteins. Generally, coronaviruses are known to be positive-stranded RNA viruses, featuring the largest viral RNA genomes known to date (27-31 kb). Seven coronaviruses have been found to cause human infection including SARS-CoV-2/2019-nCoV resulting in a potentially fatal atypical pneumonia, named COVID-19.

Like other coronaviruses, SARS-CoV-2 particles are spherical and have proteins called spikes protruding from their surface. These spikes latch onto human cells, then undergo a structural change that allows the viral membrane to fuse with the cell membrane. The viral genes can then enter the host cell to be copied, producing more viruses. Recent work shows that, like the virus that caused the 2002 SARS outbreak, SARS-CoV-2 spikes bind to receptors on the human cell surface called angiotensin-converting enzyme 2 (ACE2).

The viral genome sequence of SARS-CoV-2 was published in January 2020 and by the first week of February 2020 the crystal structure of SARS-CoV-2 main protease (PDB ID: 6LU7) was determined and released. Thereafter, the cryo-electron microscopy (cryo-EM) structure of the virus's spike protein was released. This was followed by the disclosure of several other structures of viral non-structural, structural and accessory proteins and over 360 structures of various SARS-CoV-2 proteins in the apo form or in complex with ligands and other proteins (available at www.rcsb.org).

As discussed by Arya et al. (J Mol Biol. 2021 Jan 22; 433(2): 166725) SARS-CoV-2 consists of a several nonstructural, structural and accessory proteins. The nonstructural proteins (NSPs) consist of Nsp1, Nsp3, Macrodomain-X, papain-like protease (PLpro), Nsp5/Mpro/3CLpro (a cysteine protease also known as the main protease), Nsp12, Nsp7, and Nsp8, Nsp9, Nsp10, Nsp13, Nsp14, Nsp15, and Nsp16. The structural proteins consist of the Spike protein (S) with subunits 51 and S2, nucleocapsid protein (N), membrane (M) and envelope (E) proteins. The accessory proteins consist of ORF3a, ORF7a, ORFS, and ORF9b.

It is thought that SARS-CoV-2-specific antibody responses target two proteins: the nucleocapsid protein (N) and the spike protein (S). Furthermore, it has been suggested that IgG antibodies targeting the S protein are more specific, while those targeting N may be more sensitive, particularly in the early phase of infection.

In an embodiment, the methods and devices of the invention comprise the detection of coronavirus nucleoproteins. Nucleoproteins comprise proteins that are structurally associated with DNA or RNA, and may include nucleosomes, ribosomes and nucleocapsid proteins. The terms nucleoproteins and nucleocapsid proteins are used interchangeably herein. Nucleoproteins are produced in high abundance during infection and are highly immunogenic. In the viral particle, nucleoproteins are involved with grouping the positive strand of the viral RNA and as such nucleoproteins are essential for virion assembly most likely by packaging viral RNA into helical ribonucleocapsid (RNP) and interacting with other structural proteins during virions' assembly leading to genome encapsidation. These proteins also enhance subgenomic viral RNA transcription efficiency and viral replication. Recent studies have shown that nucleoproteins are typically located in the cytoplasm and uniformly throughout the subnuclear and the nucleolus of the infected cells. Nucleoproteins are involved in several functions ranging from the formation of the viral core to virus translation, transcription and replication. Coronavirus nucleoproteins are implicated in both virus-infected primary cells and cells transfected with the plasmid express nucleoproteins protein. The SARS-CoV-2 nucleoprotein consists of two highly conserved domains: the N-terminal RNA binding domain (N-NTD; 46-174) and the C-terminal dimerization domain (N-CTD; 247-364) separated by an intrinsically disordered and highly phosphorylated linker region rich in serine/arginine (184-196, SR motif). The N- and C-terminal ends of the protein (residues 1-42 and 365-419) are disordered (Kang S. Acta Pharmaceutica Sinica B. 2020;10:1228-1238. doi: 10.1016/j.apsb.2020.04.009).

Not wishing to be bound by the following theory, it is thought that coronavirus nucleoproteins interact with fibrillarin and nucleolin. Nucleolin and fibrillarin are both major components of nucleolus. Fibrillarin protein is responsible for ribosome assembly and is essential for cell cycle regulation. It has been suggested that the interaction between nucleoprotein and fibrillarin affects ribosomal biogenesis and eases viral mRNA translation. Furthermore, it is believed that the interaction between fibrillarin, nucleolin and the viral nucleoprotein delays cytokinesis of the host cells and block the cell cycle at interphase phase, thereby allowing the virus to translate as much viral mRNAs as possible.

Viral isolation and a number of methods for detection of viral antigens, nucleic acids, and antibodies (serology) are the fundamental techniques used for the laboratory diagnosis of viral infections. Viral isolation by means of cell culture is virtually always performed in designated virology laboratories. Other methods may be performed in those laboratories as well but may also be performed in diverse laboratory sections such as general microbiology, serology, blood bank, clinical chemistry, pathology, or molecular virology. In the case of COVID-19, there is a serious and urgent need for diagnostic testing to be done outside of traditional laboratories with a growing need for rapid, easy-to-use testing in locations such as homes, schools, and businesses without the need for laboratory processing. One of the most commonly used detection assays for viral antigens involves the use of enzyme-linked immunoassays (EIAs). EIAs are available in many formats, including for example lateral flow immunoassays. Lateral flow immunoassays may also be referred to as lateral flow tests (LFT), lateral flow devices (LFD), lateral flow assays (LFA), lateral flow immunoassays (LFIA), lateral flow immunochromatographic assays, dipstick tests, express tests, pen-side tests, quick tests, rapid tests, test strips. As used herein, lateral flow immunoassays are intended to include each of the preceding terms and other such terms known to those skilled in the art.

Lateral flow immunoassays (LFIAs) are typically simple to use diagnostic devices used to confirm the presence or absence of a target analyte, such as pathogens or biomarkers in humans or animals, or contaminants in water supplies, foodstuffs, or animal feeds. LFIAs typically contain a control line to confirm the test is working properly, along with one or more target or test lines. They are designed to incorporate intuitive user protocols and require minimal training to operate. They can be qualitative and read visually, or provide data when combined with reader technology. Lateral flow tests are widely used in human health for point of care testing. They can be performed by a healthcare professional or by the patient, and in a range of settings including the laboratory, clinic or home. In the medical diagnostic industry, there are strict regulatory requirements which must be adhered to for all products developed and manufactured. LFIAs generally use immunoassay technology comprising the use of nitrocellulose membranes, colored nanoparticles, and antibodies to generate results.

LFIAs are generally designed as follows: (1) a sample pad acts as the first stage of the absorption process, and in some cases contains a filter, to ensure the accurate and controlled flow of the sample; (2) a conjugate pad, which stores the conjugated labels and antibodies, receives the sample. If the target is present, the immobilized conjugated antibodies and labels will bind to the target and continue to migrate along the test; (3) as the sample moves along the device the binding reagents situated on the nitrocellulose membrane will bind to the target at the test line. A colored line will form and the density of the line will vary depending on the quantity of the target present. Some targets may require quantification to determine target concentration. This is where a rapid test can be combined with a reader to provide quantitative results; (4) the sample will pass through the nitrocellulose membrane into the absorbent pad. The absorbent pad will absorb the excess sample. The specification of the absorbent pad will have an impact on the volume of sample the test can incorporate. Some samples require a running or sample buffer to aid sample delivery whereas other samples such as blood, serum, urine, or saliva may be able to be placed directly onto a test. Additional examples and descriptions of LFIAs are provided in U.S. Pat. No. 8,399,261 and US Patent Application No. 20070087450, each of which is incorporated herein in its entirety.

Despite the seemingly simple and ubiquitous presence of LFIAs, a high degree of immunological knowledge, engineering and technical skill is required in assembling the correct components in order to successfully create a test that is able to produce accurate, sensitive and rapid results. Tests must be customized to accommodate and process a variety of samples. In addition, tests must be designed such that the optimal binding partner(s) (i.e. antibody/antibodies) are selected to capture the maximum amount of analyte or antigen being detected. Even if all the appropriate components of the test are properly assembled, nuances in chemical processes involved in fitting the pieces together (for example, conjugating nanoparticles and antibodies) may present obstacles to creating an effective and successful testing device. The inventors herein have overcome such hurdles and designed an LFIA that accurately detects antigens associated with COVID-19 and yields rapid results.

In designing the novel LFIAs of the invention, a unique combination of nanoparticles, conjugation methods, antibodies, lateral flow assay technologies, target detection methods and antibody selection considerations were implemented.

In an embodiment of the invention, nanoparticles are conjugated to one or more detection antibodies and are deposited on a pad made of materials known to those skilled in the art such as glass fiber, cellulose fiber, nitrocellulose, polycarbonate and the like. In certain embodiments, the conjugate pad is comprised of polyester fibers and the sample pad is comprised of cellulose. Selecting the appropriate membrane involves the consideration of numerous factors, including but not limited to compatibility of the materials and properties interacting with the reagents being used, the nature of the sample (i.e. viscosity), and test goals such as sensitivity, specificity and test duration.

In an embodiment of the invention, nanoparticles are selected based on their conjugation properties to both the pad and the antibody/antibodies being utilized to capture the analyte. Suitable nanoparticles include those constructed from colloidal gold, latex and cellulose and they may be present in a variety of shapes such as, but not limited to, spheres, beads, or rods. The size range of the nanoparticles varies from 20 nm-400 nm. In certain embodiments, colloidal gold particles are used. In certain other embodiments, latex labels which can be tagged with a variety of detector reagents such as colored or fluorescent dyes, and magnetic or paramagnetic components are used. As latex can be produced in multiple colors, it has an application in multiplex assays, which require discrimination between numerous lines. Carbon and fluorescent labels, or enzymatic modification of the labels, may also be used to improve the sensitivity of the assay. In certain embodiments nanoparticles constructed of cellulose are used. The lateral flow assay technology utilized resulted in the selection of nanoparticles that generate a visually detectable signal by the eye that can be used with or without a reader for interpretation.

Conjugation of the antibody or antigen to the nanoparticle comprises specific consideration of using covalent or passive forces and techniques to bind an antibody (or antigen) to a nanoparticle. In certain embodiments, a “passive” technique is used, involving the optimization of antibody and particle ratio. In certain embodiments, a “covalent” technique is used, involving the optimization of antibody/particle ratio and EDC/NHS ratios. In certain embodiments for NHS, the antibody to nanoparticle ratio comprises 0.6:1, 0.8:1, 1:1, 1.2:1, or 1.3:1. In certain embodiments for EDC, the antibody to nanoparticle ratio comprises 1:500, 1:750, 1:1000, 1:1500, or 1:2000.

In an embodiment, the aspect of “target detection”, referring to the method of embedding the biological materials (i.e. antibody/antigen/nanoparticles/other chemicals) on a test strip is taken into consideration. One option under this consideration is “Line vs. Spot” wherein capture the antibody/antigen can be deposited on a membrane in the form of a line or a spot. Another option for target detection comprises “Singlex vs. Multiplex” referring to the number of biological targets being detected on a test strip. In an embodiment, the novel test of the invention tests for COVID-19 only (singlex). In alternative embodiments, the novel test of the invention may be modified to detect additional diseases by including additional detection lines for example for Flu A+Flu B+COVID-19 on a single test strip.

In an embodiment, the novel tests of the invention comprise COVID specific antibodies. The antibodies may be specific for any aspect or component of SARS-CoV-2, including structural, non-structural or accessory proteins associated with the spike, membrane, envelope or nucleocapsid protein. In an embodiment, the antibodies utilized in the test target the N-terminal of the nucleocapsid protein of SARS-CoV-2. In an embodiment, a single type of antibody is used. In an embodiment, more than one antibody targeting the nucleocapsid protein of SARS-CoV-2 is used. In an embodiment, more than one type of antibody, in specific and predetermined ratios is utilized. In an embodiment, a mixture of antibodies targeting various components of SARS-CoV-2 spike, membrane, envelope or nucleocapsid protein, are used. In an embodiment, the novel test and assay systems claimed herein may comprise either use a single 1+1 methodology of 1× capture antibody+1× detection antibody or multiple capture+detection antibodies (for example 2+2).

In an embodiment, the antibodies utilized in LFIAs are commercially available from a variety of sources including but not limited to: HyTest (Turku, Finland), InvivoGen (California, USA), BioRad (California, USA), Novus Biologicals (Colorado, USA), and Meridian Life Sciences (Tennessee, USA). In certain embodiments the antibodies utilized in the LFIAs comprise one or more antibodies that bind to the N-terminal part of the nucleoprotein (for example N47-A173 or N46-A174) selected from the group consisting of (Cat.# 3CV4, clones C503, C508, C510, C516, C517, C518, C524, C525, C526, C527, C528, C529, C706, C715). In certain embodiments, the antibodies utilized in the LFIAs comprise one or more antibodies selected from the group consisting (Cat.# 3CV4, clones C518, C524, C527, C706, C715) (HyTest), in certain embodiments, the antibodies comprise one or more antibodies selected from the group consisting of (Cat.# 3CV4, C706 and C715). In certain embodiments, the antibodies are selected from the group consisting of B3451M and B3449M. In certain embodiments, wherein more than one antibody is utilized, the mixture may consist of varying ratios, including but not limited to: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 2:3, 2:5, 2:7, 2:9, 3:4, 3:5, 3:7, 3:8, 3:10, 3:20, 3:40, 3:50, 3:70, 3:100, 4:5, 4:7, 4:9, 4:30, 4:50, 4:70, 4:90, 5:6, 5:7, 5:8, 5:9, 5:12, 5:16, 5:17, 5:19 amounts in between and other ratios.

In an embodiment, the novel tests of the invention have utility for detecting SARS-CoV-2 antigens including antigens of SARS-CoV-2 variants such as, but not limited to, variants of interests (Eta, Iota, Kappa), variants of concern (Alpha (B.1.1.7), Beta (B.1.351, B.1.351.2, B.1.351.3), Delta (B.1.617.2, AY.1, AY.2, AY.3), and Gamma (P.1, P.1.1, P.1.2) variants) and variants of high consequence.

a. Names for a Lateral Flow Test

In an embodiment, the present disclosure provides a novel SARS-CoV-2 Antigen Rapid Test Kit comprising a single-use qualitative lateral flow immunoassay to detect circulating antigens of SARS-CoV-2 which cause Coronavirus Disease 2019 (COVID-19). The novel assay is a point-of-care (POC) test intended for use with nasopharyngeal or nasal specimens from individuals suspected of COVID-19 infection. In an embodiment, the SARS-CoV-2 Antigen Rapid Test described herein is used as an aid in the diagnosis of SARS-CoV-2 in patients suspected of infection in combination with clinical and other laboratory test results.

In an embodiment, the rapid antigen tests described herein enable the detection of SARS-CoV-2 antigens such as those that are typically detectable in nasopharyngeal or nasal swabs during the acute phase of infection.

The present disclosure and claimed embodiments are based in part on the discovery of unexpected properties of components of the novel detection assay.

Provided herein methods and devices for detecting the presence or absence of SARS-CoV-2 antigens in mammalian samples (such as in nasopharyngeal or nasal swabs), comprising a solid support and one or more antibodies immobilized onto the solid support, wherein the one or more antibodies are capable of specifically binding to the nucleocapsid protein (or active fragment thereof) of the coronavirus. In certain embodiments, the antibodies are bound to nanoparticles. In certain embodiments, the antibodies comprise C503, C508, C510, C516, C517, C518, C524, C525, C526, C527, C528, C529, C706, C715, B3451M and B3449M. In certain embodiments, the detection device comprises a lateral flow assay.

The present disclosure provides a novel test for the detection of coronavirus and is also referred to herein as the Maxim SARS-CoV-2 Antigen Rapid Test Kit. The test kit comprises a single-use, point-of-care, chromatographic immunoassay for verification of COVID-19 diagnosis. Results can be obtained within 15 minutes. The Maxim SARS-CoV-2 Antigen Rapid Test Kit is comprised of a sample collection device (nasopharyngeal or nasal swab), Sample Buffer (Ampoule), Sample Buffer Tube & Stand, Dipstick Test Strip and Sample Labels. The Dipstick is composed of several materials which, in combination, are capable of detecting SARS-CoV-2 antigens.

In accordance with the recommended procedures for using the test claimed herein, the specimen is collected with a nasopharyngeal swab through conventional clinical procedures. The swab containing the specimen is either added directly into the Sample Buffer or placed in VTM/UTM (viral transport media/universal transport medium) for transport. Once the specimen is added to the Sample Buffer via direct insertion or VTM/UTM transfer, the dipstick is then added into the tube. The Sample Buffer and specimen mixture is absorbed through the sample pad to initiate the test run via capillary action. This sample mixture continues to migrate up the dipstick by capillary action, until it rehydrates the red colored conjugate. The sample mixture liquid will continue to move up the dipstick across a nitrocellulose membrane containing two reagent lines (in order of sample contact: Test Line and Control Line). If SARS-CoV-2 antigen is present in the sample, it will bind to the anti-SARS-CoV-2 antibodies that have been conjugated to the cellulose nanoparticles and then be captured on the test line, forming a red colored line. This indicates a SARS-CoV-2 antigen positive test result. The sample mixture liquid will continue to move up the dipstick and will bind to the control line, forming a red colored line, to indicate the test was run correctly. This built-in procedure control establishes assay validity. A red colored line in the control line region of the dipstick indicates that the dipstick functioned properly. This control line will appear on all valid tests whether the test line gives a reactive or non-reactive result. If a red colored control line does not appear, the test is invalid, and the specimen must be retested. The liquid will continue to be drawn up to the absorbent pad of the dipstick until the color on the membrane has cleared within 15 minutes after the start of the test.

The results of the test are interpreted at 15 minutes; however, results may appear prior. Results should not be read after 30 minutes. In all cases, the color intensities of the control line and test line, do not necessarily correlate to the amount of antigen captured.

In certain embodiments, the test kits claimed herein are provided in varying quantities an comprise the following items:

| | |
|---|---|
| Catalog No. | 92020-25 |
| Number of Test/Kit | 25 |
| Reagent/Material | |
| Dipstick Test Strip | 25 |
| Sample Buffer Tube | 25 |
| Tube Stand | 25 |
| Sample Buffer Ampoule | 25 |
| Nasal Swab | 25 |
| Transfer Pipette | 25 |
| Sample Label Sheet | 25 |
| Quick Reference Instruction (QRI) | 1 |
| IFU | 1 |

Additional materials useful for utilizing the tests described herein include the following: timer, personal protection equipment, and biohazardous waste containers. In an embodiment, the test kits are stored at 4-30° C. until the stated product expiration date and users are advised to allow the test kits to come to room temperature (15-30° C.) before using.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are illustrative only, since alternative methods can be utilized to obtain similar results.

### Example 1

### Sample Preparation

The test kits of the present disclosure are suitable for use with nasopharyngeal (NP) specimens. The specimen collection and test materials should be collected and available, furthermore, it is recommended that the collected specimens should be tested as soon as possible after collection. Specimens may be stored at 2-8° C. in transport medium for up to 3 days or frozen at −20° C. for long-term storage. If using frozen specimen, avoid more than 3 freeze-thaw cycles and allow to come to room temperature prior to use.

### Example 2

### Swab Test Procedure (Nasopharyngeal Swab)

Ensure test is performed in a brightly lit area for accurate result interpretation.

Ensure appropriate PPE (gloves, lab coat, face mask, and eye protection) is on and used throughout testing.

Step 1 Label the empty 4Sample Buffer tube with a unique Specimen ID and place into a tube stand.

Step 2 Take the Sample Buffer ampoule and twist off the cap. Dispense all the Sample Buffer from the ampoule into the provided Sample Buffer tube. Discard ampoule.

Step 3 Collect nasopharyngeal (NP) specimen by using the provided Nasopharyngeal Swab. To collect a nasopharyngeal swab sample, carefully insert the swab into the nostril that presents the most secretion under visual inspection. Keep the swab near the septum floor of the nose while gently pushing the swab into the posterior nasopharynx. Rotate the swab several times, then remove.

Step 4 While holding the Sample Buffer tube at a slight angle, submerge the swab with collected specimen into the Sample Buffer and thoroughly mix for 30- 60 seconds. After mixing, break the swab handle at the swab's breakpoint by bending back and forth and discard handle. Place the tube back into the tube stand.

Step 5 Set up a 15-minute timer.

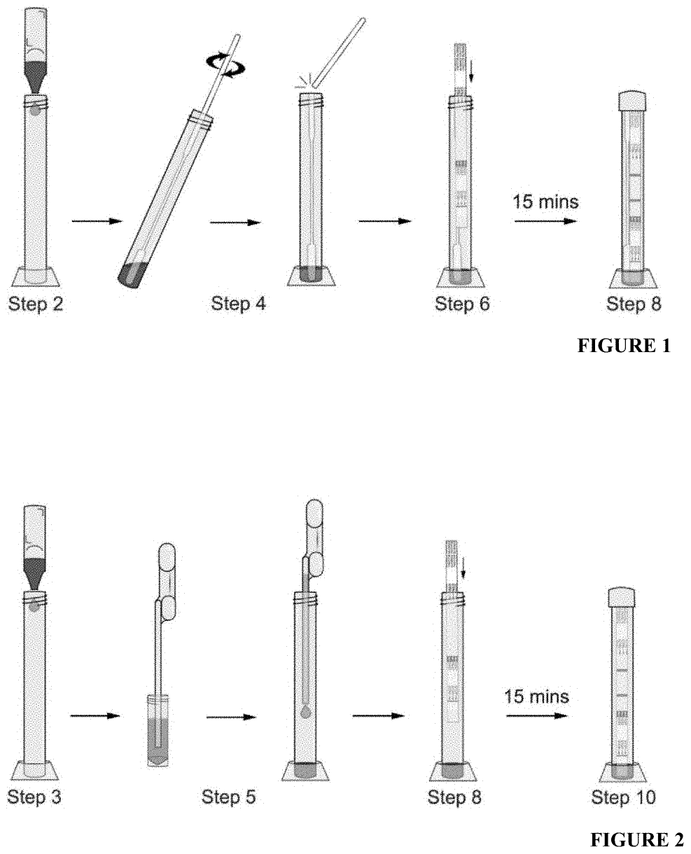

Step 6 Open and remove a Dipstick Test Strip from the foil pouch. Hold the top of the Dipstick, as indicated by “MaximBio COVID-19” and insert directly into the prepared tube (see FIG. 1). Do not allow the liquid to pass the “STOP” line; retest if necessary. Screw the tube cap on tightly.

Step 7 Start timer or if timer is not available, note the start time.

Step 8 Wait 15 minutes for test results to appear. A positive test result may take the full 15 minutes or appear sooner. Do not read after 30 minutes. Visually interpret results through tube if possible. If bands are faint, gently grip the Dipstick and pull it out so test results are exposed for clearer interpretation of results. Take precaution to ensure specimen liquid does not spill out when removing Dipstick. Return Dipstick into tube and screw cap back on. Darkness of the bands may vary. Interpret control and/or test lines even if the bands are faint. Do not interpret test results based on darkness of the bands. Refer to Interpretation of Results section below and record results.

Step 9 Ensure testing tube is securely capped and discard into biohazard container. Discard used gloves and disposable PPE into biohazard container. Sanitize any surfaces.

### Example 3

### VTM Test Procedure

Ensure test is performed in a brightly lit area for accurate result interpretation.

Ensure appropriate PPE (gloves, lab coat, face mask, and eye protection) is on and used throughout testing.

Step 1 For fresh specimens, skip to step 2. If frozen, thaw specimen at room temperature.

Step 2 Label the empty Sample Buffer tube with a unique Specimen ID and place into a tube stand. Step 3 Take the Sample Buffer ampoule and twist off the cap. Dispense all the Sample Buffer from the ampoule into the provided Sample Buffer tube. Discard ampoule.

Step 4 Shake or vortex the VTM mixture for 5-10 seconds.

Step 5 Collect 400 μL of the VTM specimen with a provided transfer pipette or calibrated pipette and empty contents into the labeled tube. To fill the transfer pipette with the VTM specimen:

1. Firmly squeeze the top bulb.

2. While still squeezing, place the pipette tip into the VTM specimen.

3. Release pressure on the bulb to fill pipette with VTM specimen.

Step 6 Screw the tube cap on tightly. Shake or vortex the tube with the added VTM specimen for 5-10 seconds. Place the tube back into the tube stand.

Step 7 Set up a 15-minute timer.

Step 8 Open and remove a Dipstick Test Strip from the foil pouch. Remove the Sample Buffer cap and hold the top of the Dipstick, as indicated by “MaximBio COVID-19”. Insert Dipstick directly into the prepared tube (see FIG. 1). Do not allow the liquid to pass the “STOP” line; retest if necessary. Screw the tube cap on tightly.

Step 9 Start timer or if timer is not available, note the start time.

Step 10 Wait 15 minutes for test results to appear. A positive test result may take the full 15 minutes or appear sooner. Do not read after 30 minutes.

Visually interpret results through tube if possible. If bands are faint, gently grip the Dipstick and pull it out so test results are exposed for clearer interpretation of results. Take precaution to ensure specimen liquid does not spill out when removing Dipstick. Return Dipstick into tube and screw cap back on.

Darkness of the bands may vary. Interpret control and/or test lines even if the bands are faint. Do not interpret test results based on darkness of the bands. Refer to Interpretation of Results section below and record results.

Step 11 Ensure testing tube is securely capped and discard into biohazard container. Discard used gloves and disposable PPE into biohazard container. Sanitize any surfaces.

Note: It is important to read results within the allotted time specified. If doing batch testing, the pouches can be partially opened for efficiency.

### Example 4

### Quality Control and Interpretation of Results

Quality Control: This rapid test contains a built-in control, the Control Line. The Control Line will develop if test runs properly. If the Control Line is not visible after running the test, the test is considered invalid and must be retested.

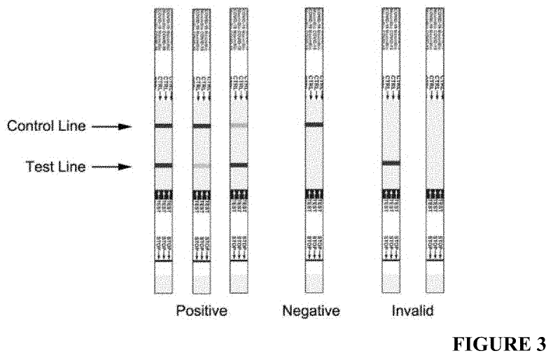

Interpretation of Results: Test Results are visually interpreted by the presence or absence of each of the two lines found on the Dipstick (see FIG. 3). The presence of any line, no matter how faint, is considered a positive result. Results obtained from this antigen rapid test should be used in conjunction with other SARS-CoV-2 diagnosis or exclusion assessments. Positive results should be considered in conjunction with the clinical history and other data available.

a. Ensure test is performed in a brightly lit area for accurate interpretation. Ensure appropriate PPE (gloves, lab coat, face mask, and eye protection) is on and used throughout testing.

b. Visually interpret results through tube. If bands are faint, carefully grip the Dipstick and gently pull out so test results are exposed for clearer interpretation of results. Take precaution to ensure specimen liquid does not spill out when removing Dipstick.

c. Following interpretation of results, appropriately discard the tube and all used materials in a biohazard waste container. Discard used gloves, PPE, and sanitize surfaces to prevent contamination prior to the performance of additional testing.

### Positive Covid-19 Infection

A sample is positive when two reactive lines appear; the Test Line and the Control Line are visible. A faint visible line located in the Test region should be considered positive. This result is consistent with an acute or recent SARS-CoV-2 infection.

False positive may occur due to cross-reacting antigens from previous infections. Samples with positive results should be confirmed with alternative testing method(s) prior to a diagnostic determination.

### Negative

A sample is negative when only the Control Line appears, and the Test Line is not visible. A negative result does not rule out SARS-CoV-2 infection and should be followed-up with a molecular diagnostic test as necessary to rule out infection in these individuals.

A test result is considered invalid if the Control Line does not appear, regardless of the presence or absence of the Test Line. A test is also invalid if no lines are present after running the test. An invalid result may indicate an inadequate or improper sample was collected, the assay was not performed correctly, or the assay is not functioning properly. Specimens that give invalid results should be retested with a new Test. If the retest still gives an invalid result, a new sample should be collected and used.

### Invalid

A test result is considered invalid if the Control Line does not appear, regardless of the presence or absence of the Test Line. A test is also invalid if no lines are present after running the test. An invalid result may indicate an inadequate or improper sample was collected, the assay was not performed correctly, or the assay is not functioning properly. Specimens that give invalid results should be retested with a new Test. If the retest still gives an invalid result, a new sample should be collected and used.

### Example 5

Material List for Chromatographic Immunoassay

Description
Sample Pad
Conjugate Pad 10 mm wide × 100 meters - 3″ plastic core
Conjugate Pad 28 mm wide × 100 meters - 3″ plastic core
Nitrocellulose Membrane - 25 mm × 100 m
80 mm Backing Cards
Cover Tape Top - “C” line indicator
Cover Tape Bottom - “Stop” + “T” line indicator
Conjugate NanoParticles
Mab to SARS-CoV-2 Nucleocapsid (NP)
Mab to SARS-CoV-2 Nucleocapsid (NP)
Goat anti Human IgG - Striping Control
Sucrose
BSA Blocking Buffer
Tween 20
Proclin 950
Sodium Chloride
Sodium Phosphate Monobasic
Sodium Phosphate Dibasic, Heptahydrate

1. A method for detecting the presence of coronavirus antigens in a sample obtained from a subject,
a. wherein the method comprises the use of lateral flow immunoassay comprising a solid support, nanoparticles and one or more antibodies,
b. wherein the one or more antibodies bind coronavirus nucleocapsid protein, spike protein, membrane protein and/or envelope protein.

2. The method of claim 1, wherein the coronavirus antigens are associated with 229E, NL63, OC43, HKU1, MERS-CoV, SARS-CoV, SARS-CoV-2.

3. The method of claim 2, wherein the coronavirus antigens are associated with SARS-CoV-2 variants selected from the group consisting of Eta, Iota, Kappa, Alpha (B.1.1.7), Beta (B.1.351, B.1.351.2, B.1.351.3), Delta (B.1.617.2, AY.1, AY.2, AY.3), and Gamma (P.1, P.1.1, P.1.2).

4. The method of claim 1, wherein the one or more antibodies bind to coronavirus nucleocapsid protein.

5. The method of claim 1, wherein the one or more antibodies comprise C503, C508, C510, C516, C517, C518, C524, C525, C526, C527, C528, C529, C706, C715, B3451M or B3449M.

6. The method of claim 1, wherein the one or more antibodies are present in ratios of 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 2:3, 2:5, 2:7, 2:9, 3:4, 3:5, 3:7, 3:8, 3:10, 3:20, 3:40, 3:50, 3:70, 3:100, 4:5, 4:7, 4:9, 4:30, 4:50, 4:70, 4:90, 5:6, 5:7, 5:8, 5:9, 5:12, 5:16, 5:17, 5:19.

7. The method claim 1, wherein the one or more antibodies comprise C518, C524, C527, C706, C715 present in a ratio of 1:1.

8. The method of claim 1, wherein the one or more antibodies are bound to nanoparticles comprising colloidal gold, latex and cellulose.

9. The method of claim 1, wherein the one or more antibodies are bound to the nanoparticles, and wherein the nanoparticles comprise cellulose nanoparticles.

10. The method of claim 8, wherein the one or more antibodies are bound to the nanoparticles via covalent binding.

11. The method of claim 10, wherein covalent binding occurs via NHS and the antibody to nanoparticle ratio comprises 0.6:1, 0.8:1, 1:1, 1.2:1, or 1.3:1.

12. The method of claim 10, wherein covalent binding occurs via EDC and the antibody to nanoparticle ratio comprises 1:500, 1:750, 1:1000, 1:1500, or 1:2000.

13. The method of claim 1, wherein the sample comprises a nasopharyngeal swab.

14. The method of claim 1, wherein the lateral flow immunoassay comprises a sample pad and a conjugate pad.

15. The method claim 13, wherein the sample pad comprises cellulose, and the conjugate pad comprises polyester fiber.

16. The method of claim 1, wherein the one or more antibodies comprise C518, C524, C527, C706, C715 present in a ratio of 1:1 bound to cellulose nanoparticles via NHS and wherein the antibody to nanoparticle ratio comprises 0.6:1, 0.8:1, 1:1, 1.2:1, or 1.3:1.

17. The method of claim 1, wherein the one or more antibodies comprise C518, C524, C527, C706, C715 present in a ratio of 1:1 bound to cellulose nanoparticles via EDC and the antibody to nanoparticle ratio comprises 1:500, 1:750, 1:1000, 1:1500, or 1:2000.

18. A device for detecting the presence of coronavirus antigens in a sample obtained from a subject,
a. wherein the devices comprises a lateral flow immunoassay comprising a solid support, nanoparticles and one or more antibodies,
b. wherein the one or more antibodies are immobilized onto the solid support, wherein the one or more antibodies are capable of specifically binding to the nucleocapsid protein (or active fragment thereof) of the coronavirus.

19. The device of claim 18, wherein the one or more antibodies comprise C503, C508, C510, C516, C517, C518, C524, C525, C526, C527, C528, C529, C706, C715, B3451M or B3449M.

20. The device of claim 18, further comprising sample collection device (nasopharyngeal swab), Sample Buffer (Ampoule), Sample Buffer Tube & Stand, Dipstick Test Strip and Sample Labels.
